(19) United States
(12) Patent Application Publication
(10) Pub. No.: US 2024/0312607 A1
(43) Pub. Date: Sep. 19, 2024

(54) Automated AI-Based Method And System For Dynamically Prioritizing Patients' Waiting Lists

(71) Applicant: SERENUS AI LTD., Tel-Aviv (IL)

(72) Inventors: Josef Elidan, Tel-Aviv (IL); Orly Elidan-Harel, Hod-HaSharon (IL)

(21) Appl. No.: 18/571,234

(22) PCT Filed: Jun. 14, 2022
(86) PCT No.: PCT/IB2022/055479

§ 371 Date: Dec. 17, 2023

Related U.S. Application Data
(60) Provisional application No. 63/211,563, filed on Jun. 17, 2021.

(51) Int. Cl. G16H 40/20; G16H 10/60; G16H 15/00; G16H 50/70; H04L 51/02

(52) U.S. Cl. CPC G16H 40/20; G16H 10/60; G16H 15/00; G16H 50/70; H04L 51/02

(57) ABSTRACT

An automated AI-based computerized system for dynamically prioritizing patients' waiting lists, comprising: a system server configured to: communicate with external medical sources; store medical information from the external medical sources in a database; and analyze the medical information using Natural Language Processing (NLP) and artificial intelligence tools; the system server comprises a machine learning module configured to: collect patients' profiles and at least one other factor; analyze the patients' profiles and the at least one other factor; and prioritize one of an appointment and a waiting list of a patient, according to the analysis of the patients' profiles and the at least one other factor.

### CROSS-REFERENCE TO RELATED PATENT APPLICATIONS

This patent application claims priority from and is related to U.S. Provisional Patent Application Ser. No. 63/211,563, filed Jun. 17, 2021, this U.S. Provisional Patent Application incorporated by reference in its entirety herein.

### FIELD OF THE INVENTION

The present invention generally relates to the field of medical systems and specifically to an AI-based method and system for dynamically prioritizing patients' waiting lists.

### BACKGROUND OF THE INVENTION

Hundreds of millions of medical treatments and procedures are carried out each year worldwide. However, health systems today are overloaded, and the resources are limited. Thus, at many times, patients wait a relatively long time for their treatment without a thorough analysis with regards to the patients' relative condition, the urgency of the treatment and the consequences of not providing the treatment at the right time, leading to poor prognosis. This paint point is expected to increase in the next few years as the population continues to grow and rapidly attains extreme proportions during extraordinary emergencies such as a pandemic, which is putting a huge burden on health systems. Therefore, innovative, high-performing technology tools are urgently needed for real-time prioritization of patients' waiting lists.

In order to efficiently prioritize waiting lists of patients waiting for their treatment(s), there is a need for advanced technologies and machine learning capabilities.

Therefore, there is a need for an automated AI-based method and system for dynamically prioritizing patients' waiting lists, thereby enabling to provide treatments based on the patient's specific relative clinical condition and additional various factors which are needed to be taken into consideration in order to efficiently prioritize patients' waiting lists.

### SUMMARY OF THE INVENTION

According to an aspect of the present invention there is provided an automated AI-based computerized system for dynamically prioritizing patients' waiting lists, comprising: a system server configured to: communicate with external medical sources; store medical information from the external medical sources in a database; and analyze the medical information using Natural Language Processing (NLP) and artificial intelligence tools; the system server comprises a machine learning module configured to: collect patients' profiles and at least one other factor; analyze the patients' profiles and the at least one other factor; and prioritize one of an appointment and a waiting list of a patient, according to the analysis of the patients' profiles and the at least one other factor.

The system server may comprise a reports and statistics module configured to generate personal reports to users following a chatbot session; wherein the chatbot may be configured to enable bi-directional communication with users seeking a prioritized appointment; and wherein the communication may comprises personally customized dynamic scenario comprising dynamic factors, responses and dynamic weights of the responses.

The system server may further comprise: a data mining and NLP module; a machine learning module; an Application Program Interface (API) module configured to enable data retrieval from various external medical sources; a management and control module; at least one database; a web application configured to provide users with an interactive platform for communicating with the system; and a processing engine.

The data mining and NLP module may be configured to extract data from the external medical sources and transform it into an understandable structure for further use.

The extracted data may comprise data from patients' medical files.

The extracted data may be used for automatic labeling, for training the machine learning module.

The machine learning module may be configured to: calibrate the dynamic weight of each response relevant to each treatment, by analyzing a large number of scenarios; and calibrate the system using at least one of: information mined from real medical files; professionals' and/or patients' feedback after having undergone a treatment; and scanning latest researches, statistics and publications by health organizations.

The at least one database may comprise: patients' personal and medical information; reports generated by the reports and statistics module; a set of specific factors and possible responses for each treatment with complex relations, which are generated in advance by human experts and/or by machine learning modules; and a set of dynamic weights associated with each response for different scenarios.

The factors and possible responses may be generated and updated by the system for each treatment, based on the data mining and NLP module and the machine learning module.

The dynamic weights may be generated and updated by the system for each treatment, based on the data mining and NLP module and the machine learning module.

The communication with the system may comprise presenting queries, receiving responses and receiving reports and recommendations.

The processing engine may be configured to: select and present one factor at a time to the user; receive a response to the factor; assign a current dynamic weight to the user's response; optionally assign a tag (key) to the user's response; select next factor based on the user's response and one or more of the optional tags assigned to the user for previous responses; and provide results to the reports and statistics module.

The at least one database may comprise, for each treatment: a set of result range objects (RRO); and a multi-dimensional tree of factor nodes (FN) and response objects (RO) for each factor node.

The RRO may comprise one or more parameters selected from the group consisting of: treatment ID, Minimum Range Value (MINRV), Maximum Range Value (MAXRV), result text and result description.

The FN may comprise one or more parameters selected from the group consisting of: factor text, factor priority, required keys for unlocking the factor, indication whether the factor has an automatic response, min and max Visual Analog Scale (VAS) range, min score to show factor; max score to show factor and a set of response objects (RO).

The RO may comprise one or more parameters selected from the group consisting of: response text, response dynamic_weight, response key, automatic keys to choose response, force score to assign to user and indication of test end.

The at least one other factor may be selected from the group consisting of: reversible indications, none reversible factors, timing, doctors' labeling, patients' outcomes including objective and subjective indicators to the selected treatment and predetermined or selected factors, according to the patient.

According to another aspect of the present invention there is provided an automated method of dynamically prioritizing patients' waiting lists, comprising: retrieving medical data from medical sources and storing the retrieved data; analyzing the medical data using Natural Language Processing (NLP) and artificial intelligence tools; collecting patients' profiles and at least one other factor; analyzing the patients' profiles and the at least one other factor; and prioritizing one of an appointment and a waiting list of a patient, according to the analysis of the patients' profiles and the at least one other factor.

The collection of the patients' profiles may comprise: computerizing a set of dynamic factors and possible responses in a hierarchic data structure with complex relations and a different dynamic weight for each response in the context of each treatment and scenario, the dynamic weights calculated by analyzing, using an artificial intelligence module, the treatment data; receiving from a user, a request to prioritize an appointment; providing a personalized customized dynamic scenario to the user, the scenario dynamically created, using the artificial intelligence module, according to the treatment, responses of the user, and the dynamic weights assigned to the user's responses to previous factors in the scenario; computing a relative indication including providing a positive impact if a specific response and its dynamic weight, supports the treatment, and a negative impact if a specific response and its dynamic weight, negates the treatment according to the response's relative importance and impact on a decision to conduct the treatment; and generating a specific personalized report for the user based on the treatment and including a relative indication for the treatment.

The artificial intelligence module may comprise a logic base derived from experience of human experts, statistical information and analysis of published studies and machine learning modules.

The method may further comprise assigning at least one key (tag) to the user's response.

The dynamical creation of the scenario may comprise selecting a next factor according to keys accumulated so far in the scenario.

The dynamical creation of the scenario may comprise ending the scenario according to keys accumulated so far in the scenario.

The user may be a patient and wherein the specific personalized report may comprise at least one of: data related to the treatment, statistics, risks and questions to ask their physicians before going under the treatment.

The user may be a medical professional and wherein the specific personalized report may comprise at least one of: data on at least some of treatments, statistics, risks and other factors with regards to treatments decision making process in the daily practice.

The at least one other factor may be selected from the group consisting of: reversible indications, none reversible factors, timing, doctors' labeling, patients' outcomes including objective and subjective indicators to the selected treatment and predetermined or selected factors, according to the patient.

According to another aspect of the present invention there is provided an automated method of dynamically prioritizing patients' waiting lists, comprising: selecting, by a patient, an appointment to be prioritized; fetching or collecting a patient's profile of the patient and other patients' profiles; analyzing the patient's profile, the other patients' profiles and at least one other factor; prioritizing one of an appointment and a waiting list of the patient, according to the patient's profile, the other patients' profiles and the at least one other factor.

The at least one other factor may be selected from the group consisting of: reversible indications, none reversible factors, timing, doctors' labeling, patients' outcomes including objective and subjective indicators to the selected treatment and predetermined or selected factors, according to the patient.

### BRIEF DESCRIPTION OF THE DRAWINGS

For better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 is a schematic block diagram of the system, according to embodiments of the present invention;

FIG. 2 is a schematic block diagram of an exemplary implementation of the structure of a single treatment entry in the system database;

FIG. 3 shows an exemplary structure of a factor node for factor No. 186;

FIG. 4 shows an exemplary structure of a response object for factor No. 186 of FIG. 3;

FIG. 5 shows an exemplary score table for an exemplary treatment—a tonsillectomy procedure;

FIG. 6 is a flowchart showing the steps taken during an exemplary session initiated by a user for obtaining second opinion regarding a medical treatment;

FIGS. 7A-7H provide an example of factors, possible responses and responses scoring, for a single treatment;

FIGS. 8A and 8B are exemplary reports provided by the system to the user at the end of a session;

FIG. 9 is a flowchart showing the process performed by the system of present invention when a personalized appointment has to be scheduled;

FIG. 10A shows an exemplary chart with the configured results to prioritize patients' waiting lists according to the ML predications and factors considered; and

FIG. 10B shows exemplary detailed information for each case prioritized.

### DETAILED DESCRIPTION OF PREFERRED EMBODIMENTS

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is applicable to other embodiments or of being practiced or carried out in various ways. Also, it is to be understood that the phraseology and terminology employed herein is for the purpose of description and should not be regarded as limiting.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire line, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The operations and algorithms described herein can be implemented as executable code within the control unit—and/or processor circuit—as described, or stored on a standalone computer or machine readable non-transitory tangible storage medium that are completed based on execution of the code by a processor circuit implemented using one or more integrated circuits. Example implementations of the disclosed circuits include hardware logic that is implemented in a logic array such as a programmable logic array (PLA), a field programmable gate array (FPGA), or by mask programming of integrated circuits such as an application-specific integrated circuit (ASIC). Any of these circuits also can be implemented using a software-based executable resource that is executed by a corresponding internal processor circuit such as a microprocessor circuit (not shown) and implemented using one or more integrated circuits, where execution of executable code stored in an internal memory circuit (e.g., within a memory circuit) causes the integrated circuit(s) implementing the processor circuit—to store application state variables in processor memory, creating an executable application resource (e.g., an application instance) that performs the operations of the circuit as described herein. Hence, use of the term “circuit” in this specification refers to both a hardware-based circuit implemented using one or more integrated circuits and that includes logic for performing the described operations, or a software-based circuit that includes a processor circuit (implemented using one or more integrated circuits), the processor circuit including a reserved portion of processor memory for storage of application state data and application variables that are modified by execution of the executable code by a processor circuit. The memory circuit can be implemented, for example, using a non-volatile memory such as a programmable read only memory (PROM) or an EPROM, and/or a volatile memory such as a DRAM, etc.

An automated method and system is provided, which provides a management tool for prioritizing patients' waiting lists. It will be appreciated that the term ‘treatment’ may include, but is not limited to, any medical procedure or any medical treatment including but not limited to surgeries, medication, cancer management and more. The method and system of the present invention takes into consideration various factors, which will be described below, in order to provide treatment to, e.g., a patient who needs a treatment with a relevant priority over another patient. The AI-based method and system of the present invention may use the patient's clinical history, the knowledge of most recent research in each field, statistics, experts' knowledge, prospective data including objective indicators of patients' outcomes, patients' and professionals' feedback, as well as machine learning technologies.

The term ‘waiting list’ is usually being used to describe a list of individuals waiting for a certain service which is not available, e.g., there are 100 appointment to a certain doctor and 200 patients, thus, the first 100 have an appointment and the rest are waiting for an appointment in a waiting list.

It will be appreciated that the term ‘waiting list’ as used hereinbelow may refer to any one of those patients, the ones that do have an appointment and the ones that do not.

Therefore, the present invention may dynamically prioritize appointments for patients who already have an appointment and for ones waiting for an appointment.

The system database is typically constantly reviewed and updated, using machine learning techniques, based on one or more of: professionals and patients' feedback, physicians and supervisors' decisions (labeling), objective patient outcomes indicators such as but not limited to: readmissions, physician visits records, complications, additional treatments provided, research and a large number of medical records, including external data received, for example, from hospitals, health maintenance organizations (HMOs) and the like. It will be appreciated that the terms ‘user’ and ‘patient’ may be used intermittently throughout the description.

The training of the system is developed from at least some of the following sources:
Research and guidelines.
Experts' Knowledge and labeling.
Historical and optionally simulated data (due to lack of sufficient historical data).
Real prospective data with regards to patients' outcomes.

The training of the system is conducted, but is not limited to be conducted, with at least some of the following stages:
1. Prioritizing patients' waiting lists according to an accumulated score of the patient and suggested groups. For example:
   Treatment is indicated (high level indication).
   Treatment is indicated (medium level indication).
   Treatment is indicated (low level indication).
   Equivocal result. Further consultation is needed.
   Reversible condition—Equivocal result. Further consultation is needed.
   Reversible condition—There is no indication for the treatment.
   There is no indication for the treatment, subject to a doctor's decision.
   Irreversible condition—Equivocal result. Further consultation is needed.
   Irreversible condition—There is no indication for the treatment.
2. Adding additional factors to be considered for the prioritization, For example:
   No indication.
   Reversible—a reversible factor such as, for example, diabetes.
   Non-reversible—no indication for the procedure.
3. Timing—the period a patient is waiting for a treatment.
4. Any other factor—which will also be learned from prospective learning and patients' outcomes to similar treatments.
5. Adding physician's labeling with regard to the appropriate prioritization. ML algorithms predict better prioritization.

6. Learning from patients' outcomes/ML algorithms—improving patients' list prioritization according to patients' outcomes (for example—pregnant women may better be delayed for better outcomes). The patients' outcomes can be learned from objective indicators such as readmissions, complications etc, as well as from subjective indicators such as patients' and physicians' feedback.

Following a solid expert system and a learned improvement in place, the system of the present invention uses real-world data to continuously improve the results using advanced learning machinery by allowing for a greater breadth of measurements, “big data” scale of data processing using advanced machine learning techniques such as random forests or deep learning. At the high level, at the context of the present invention, three axis of data and ML progression may be pursued in parallel:
1. User-Based Records. One of the benefits of putting a reasonable baseline in place is that this model can be used to provide a useful service to users (patients). By simply using the system, each user creates a real record, which can then be labeled by an expert relatively quickly. Further, user follow-up feedback can serve as a (noisy) automated labeling mechanism which can further improve the system, similar to user labeling of images in a personal photo album. The obvious benefit of this axis is the low-cost accumulation of real cases.
2. Unlabeled Medical Records. Medical records from the practices of experts and/or from medical institutions can similarly be labeled by experts. This can allow learning techniques to be applied to much richer measurement spaces, taking into account a wealth of information that goes well beyond what can be captured by a hand-coded scenario. While ultimately of higher quality than (1), this axis of progress is naturally slower and should just be pursued in parallel. Importantly, error analysis of models learned as part of (1) can act as a guide for informed collection of records, speeding up this stage.
3. Labeled Medical Records. Many health and insurance institutions also have medical records that have been labeled by the treating doctor. The obvious benefit is the availability of a large number of labeled records. While the quality of the labels is likely to be lower than that of the best physician in the field, mixture of experts' machine learning techniques can be used to mitigate this problem (often surpassing the best expert). Ultimately this fully automated axis is likely to lead to the biggest progress, simply due to the high-volume of (reasonably) labeled examples.

Emphasis shall be on collecting objective indicators with regards to patient outcomes, such as, but not limited to, readmissions, complications, visits, patients' feedbacks and professionals' feedbacks.

Methods of finding insights between the data collected from historical data or the personalized chatbot, treatment protocols, the patient outcomes and the prioritization given shall be implemented.

The system operates, but is not limited to operate, on a number of levels:
1. Storing detailed anonymous medical information relating to the patient.
2. Providing a detailed report, including patient's responses to factors presented in a personalized chatbot, detailing the relative impact (positive or negative) of each factor that contributed to a recommendation before a contemplated medical treatment (e.g., additional tests or treatments).
3. Performing statistical analysis and finding hidden correlations between variables of multiple patients by interfacing with large medical systems (big data) and acquiring large volumes of prospective information referring to patient outcomes to be processed by the system's machine learning modules.
4. Improving the accuracy of the system by using machine learning modules and by analyzing a large number of patients' records (big data) and users' feedback (prospective data).
5. Adding new indications for treatment by using machine learning modules and by analyzing a large number of patients' records and users' (physicians and patients) feedback.
6. Using machine learning modules for finding correlations between patients' anonymous profiles (via chatbot or medical records), treatment protocols and patient's outcomes as results of treatments (objective indicators), thereby predicting treatment efficacy.
7. Using machine learning modules for finding correlations between at least some of the above levels (1-6), thereby enabling to dynamically and effectively prioritizing waiting lists.

The system is configured to be installed in, amongst others but not limited to:
Medical institutions and HMOs, health systems, to be used as an assisting tool to effectively prioritize patients waiting lists.
Health Insurance companies, to be used as a filtering tool before and after authorizing a treatment.
A proprietary website, to be used by patients seeking a medical appointment.

The system uses a number of novel technologies, including:
1. A dynamic algorithm for building a personalized set of factors responses with a dynamic weight for each factor and an accumulated weight to create a personalized chatbot to collect patients' anonymous data in order to dynamically prioritize patients' waiting lists.
2. A machine learning system that determines and improves the weight/impact of each relevant factor for the treatment being considered in order to dynamically prioritize patients' waiting lists.
3. A machine learning system that interfaces with medical records and objective patient outcomes indicators such as and not limited to readmissions, complications etc. and professionals' and patients' feedbacks for the purpose of finding new indications and hidden correlations between data of a plurality of patients regarding the recommendations given for treatments, thereby enabling to dynamically prioritize patients' waiting lists.

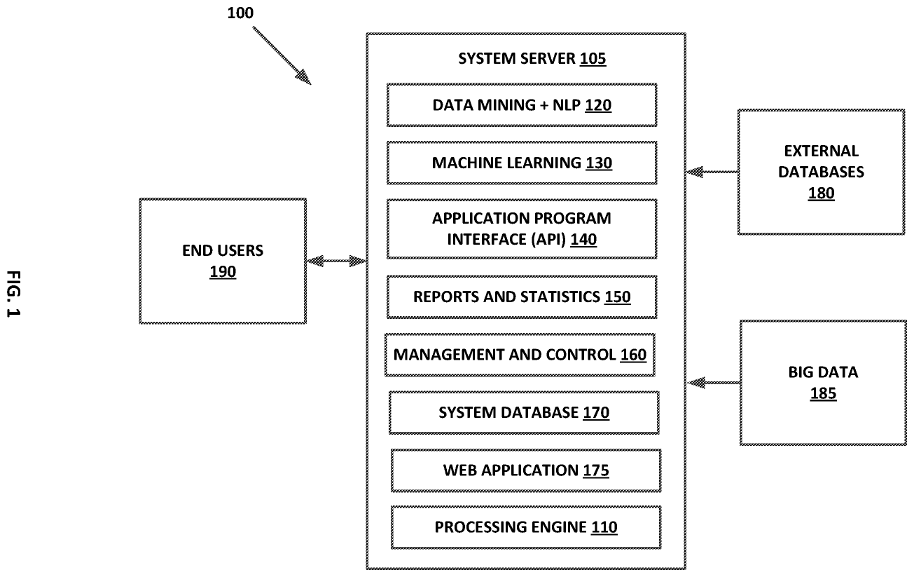

FIG. 1 is a schematic block diagram of the system, according to embodiments of the present invention. System 100 comprises one or more system servers (only one shown) 105, preferably a web server, communicating over the Internet with external databases 180, such as medical institutions' databases comprising patients' files, with big data resources 185, including both structured and unstructured data and with end users' electronic communication means 190, such as medical institutions' systems and patients' computers and/or mobile electronic communication devices.

System server 105 comprises a processor and some or all of the following computerized modules:
A data mining and Natural Language Processing (NLP) module 120, configured to extract information from external databases 180 and transform it into an understandable structure for further use, using NLP techniques. Data extracted includes, for example, data from patients' medical files such as lab reports, free text notations etc. The extracted data is used for automatic labeling for training the machine learning module.
A machine learning module 130, configured to some or all of:
   Calibrate the weight (impact) of each variable relevant to each treatment, by analyzing a large number of scenarios.
   Calibrate the system using information mined from prospective real medical files (Big Data).
   Calibrate the system using professionals and patients' feedback after having undergone the treatment.
   Calibrate the system by scanning latest researches, statistics and publications by health organizations (e.g., American Academy Guidelines, World Health Organization, American and European health organizations, etc.).
   Find connections and correlations between various factors, such as, for example, patients' profiles and patients' outcomes as results of treatments for dynamically personalize waiting lists using some or all of patients' data and reports received from at least one of the data mining and (NLP) module 120, the external databases 180, the big data resources 185 and the reports and statistics module 150.
An Application Program Interface (API) module 140 configured to enable data retrieval from various external medical sources.
A reports and statistics module 150 configured to generate personal reports to patients following a factor and response session and to provide statistics calculated from a plurality of reports.
A management and control module 160 configured to manage the system including managing fields, treatments, factors and possible responses, databases, scores spectrums, impacts of responses, export and import information for machine learning purposes, managing clients, managing a combination of treatments. Managing-adding, editing and removing records. The management system is rule and permissions based and is constantly being updated and evolved.
One or more database 170, storing:
   Patients' anonymous clinical information including lab test results, anamnesis and reports generated by the reports and statistics module 150;
   A set of specific queries and possible responses for each treatment, which are generated in advance, e.g., by human experts;
   A set of weights associated with each factor, which are pre-determined according to general knowledge and continuously and dynamically updated by the latest researches, statistics and guidelines of the American and European Academies, and by machine learning modules 130;
   Connections and correlations between patients' profiles as extracted from medical records or input by an interactive chatbot, treatment protocols and patients' outcomes as results of treatments for dynamically prioritizing waiting lists.
A web application 175, providing users with an interactive platform for communicating with the system over the Internet, including presenting queries, receiving responses, receiving reports and recommendations and a providing an appointment for treatment.
A processing engine 110, configured to:
   Select and present one query at a time to the user;
   Dynamically grade user's responses according to currently associated weight, based on machine learning modules;
   Determine next query based on last response and the accumulated weight and optionally the weight of the previous response;
   Dynamically update weights according to previous and following responses and associated weights.
   Provide results to reports and statistics module 150.

Typically, in a set-up phase, a set of specific factors for each patient and treatment are generated in advance, e.g., by human experts.

The factors are organized in a hierarchic flowchart in complex relations and each response receives a different weight (impact) according to a specific scenario, the previous factor and weight and the accumulated weight and treatment which, as said above, may be dynamically updated throughout the process, based on machine learning modules.

The hierarchical flow and weights (impacts) may be organized in any suitable logical relationship or structural combination. It is possible that each case shall receive a different set of factors and that different weights may be assigned to the same factors in different combinations, e.g., so as to yield a specific output for each individual end-user.

As a result and during the process, each user views a personally customized dynamic scenario, according to the selected assigned treatment, and the user's responses.

At the end of the process, the weights received or updated for all the responses are analyzed to provide the relevant output.

According to certain embodiments, a patient facing a particular treatment is asked relevant factors regarding his medical condition.

According to embodiment of the invention, some responses may be pulled automatically by the system from the patient's medical records using NLP techniques.

Each response receives a certain dynamic impact value, according to the relative importance and impact (i.e., weight) on the decision to conduct the specific treatment. If the response negates the treatment, it receives a negative impact.

At the end of the process the system analyzes the input and the patient and/or the medical specialists receives a result, with the relative indication or contraindication for the treatment and additional recommendations, e.g., further tests and conservative treatments needed before undergoing the treatment.

Examples of results according to which the patient is being prioritized (dynamic):
Low indication for the treatment.
Moderate indication for the treatment.
High indication for the treatment.
Equivocal results. A second opinion or further discussion is needed.
The treatment is not justified.
The process is terminated because some crucial information is missing.
The process is terminated because more evaluation (test) is needed.

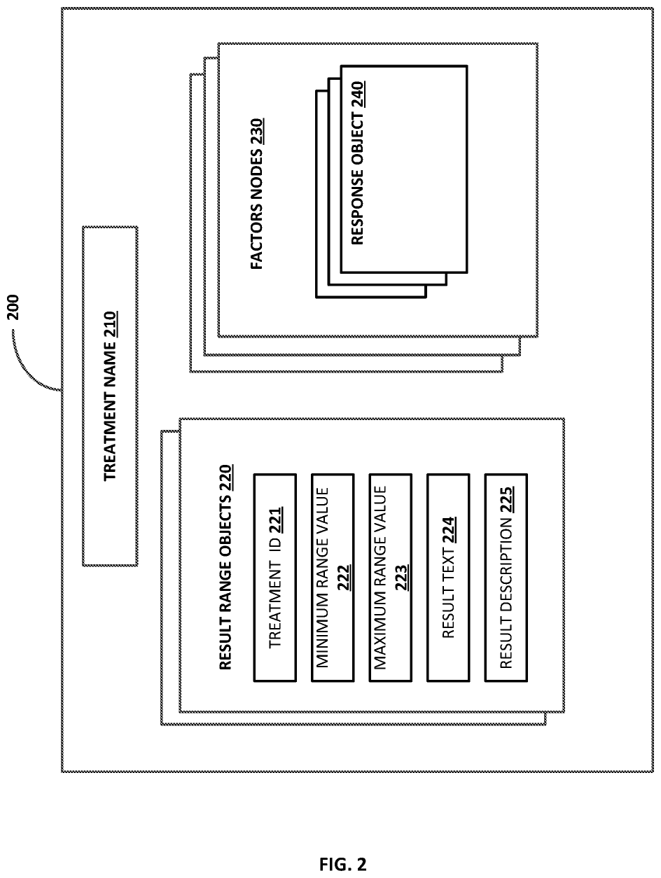

FIG. 2 is a schematic block diagram of an exemplary implementation of the structure of a single treatment entry 200 in the system database 170.

A treatment entry 200 may comprise some or all of the following objects:
Treatment name 210;
A set of result range objects (RRO) 220;
A multi-dimensional tree of factor nodes (FN) 230 and response object 240 for each factor node.

A Result Range Object (RRO) 220 may for example comprise some or all of the following objects:
Treatment ID 221;
Minimum Range Value (MINRV) (number) 222;
Maximum Range Value (MAXRV) (number) 223;
Result Text 224;
Result Description 225.

A factor node (FN) 230 may for example comprise some or all of the following objects:
Factor text (text);
Factor priority;
Required keys for unlocking the factor (optional);
Indication whether the factor has an automatic response;
Visual Analog Scale (VAS) range (min and max);
Min score to show factor;
Max score to show factor;
A set of response objects (RO).

A response object (RO) 240 may for example comprise some or all of the following objects:
Response text (text);
Response weight (integer-positive or negative);
Response key (AK) or any other indicator for automatically selecting the next factor node.
Automatic keys to fire response;
Force score to assign to user;
Indication of test end.

The keys serve for tracking selected ones of the user's responses and may change the course of the session by selecting the next factor, determining whether the session should terminate and force a score.

The keys method may be replaced by any other method for automatically identifying which factors are relevant to a particular context, given a collection of possible factors that are relevant to a treatment, the factors dynamic weight and the accumulated weight.

Three components, together or independently, contribute to these methods:
1) An expert and/or ML prediction prior indicating the relevancy of a particular factor given a particular response to another factor.
2) An expert label that is generated as follows: factors are presented to the expert, either one after the other or as a complete scenario and the expert evaluates the relevancy of each factor to the given context (either as a numerical score or a categorical one, e.g., “relevant”, “not relevant”).
3) Factors are identified as irrelevant based on statistical evaluation of relevancy to the target diagnosis task from past records.

The learning system may use either one or all of these components to learn a unified prediction system for the relevancy of a factor in a particular context. This relevancy “score” will be used to automatically modify the structure of the scenario.

Another method that may replace keys:

Factor tree—a data structure comprising nodes or factors and responses nodes in which each factor is a node and has a parent response.

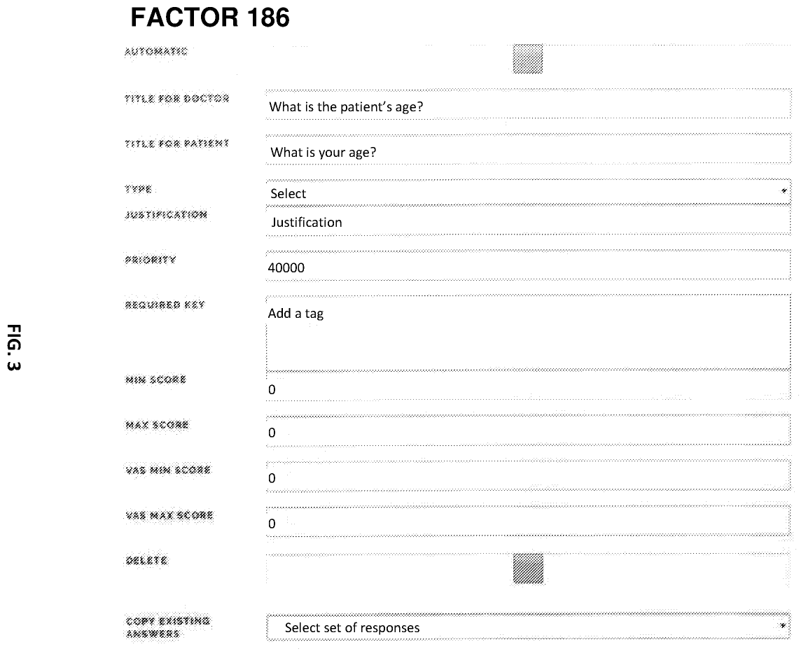

FIG. 3 shows an exemplary structure of a factor node 230 for factor No. 186.

The factor node comprises interactive fields, which may be initially filled by a medical expert and later manually modified by a system administrator or automatically modified by the machine learning module 130.

The fields in the factor node of FIG. 3 are:
Automatic—selection of the Automatic mode means that the factor is an automatic factor defined in the system and not presented to the system users. Automatic factors are actually “if—then” clauses that examine a set of tags (keys) assigned to the user during a session and determine a next course of action for the test accordingly.
Title for doctor—the factor text to be presented to a user who is a physician.
Title for patient—the factor text to be presented to a user who is a patient.
Type—selection between a number of types assigned by the system to the factor. The selection is made according to the impact of the factor on the treatment. Exemplary selection items are: main treatment factor, Critical examination, general health, etc.
Justification—free text.
Priority—priority assigned to the factor. The priority influences the order of factors presented during a session.
Required key—keys (tags) that should have been assigned to the user during the current session in order to present this factor.
Min score—the minimal score required to be accumulated during the current session in order to present this factor.
Max score—the maximal score allowed to be accumulated during the current session in order to present this factor.
VAS min score—a number to which a minimal score given in a Visual Analog Scale (VAS) response should be transformed.

VAS max score—a number to which a maximal score given in a Visual Analog Scale (VAS) response should be transformed.
Delete—selection of the Delete option means that the factor is to be deleted from the system.
Copy existing responses—selection between response sets to be copied from other factors.

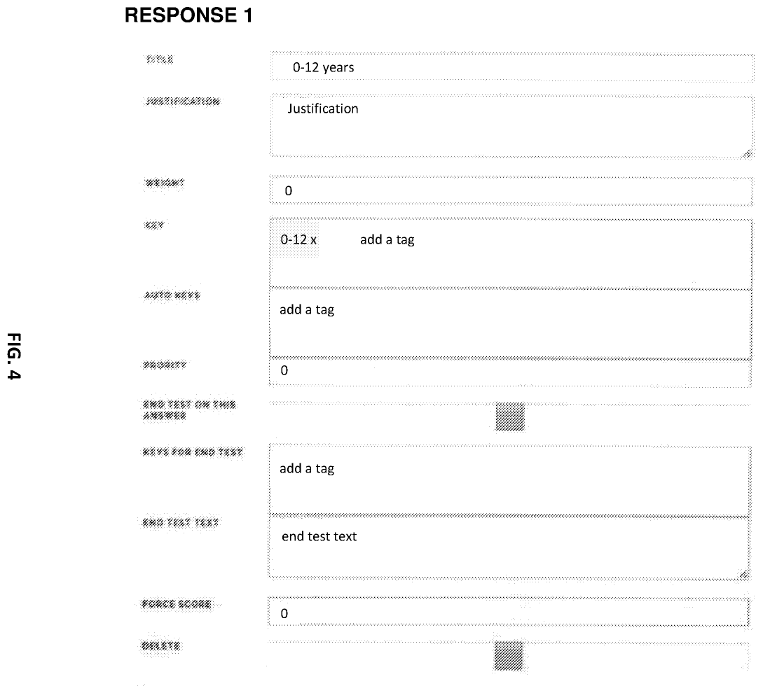

FIG. 4 shows an exemplary structure of a response object 240 for factor No. 186 of FIG. 3.

The response object comprises interactive fields, which may be initially filled by a medical expert and later manually modified by a system administrator or automatically modified by the machine learning module 130.

The fields in the response object of FIG. 4 are:
Title—the text selected by the user (optionally, out of a plurality of selectable responses).
Justification—free text.
Weight—the weight (impact) assigned by the system to the current response in the context of the treatment examined by the current session.
Key—the key (tag) assigned by the system to the current response.
Auto keys—optional one or more keys defined by the system in order to assign an automatic response to the factor if the factor is an automatic factor.
Priority—priority assigned to the response, meaning in which order the possible responses shall appear to the user.
End test on this response—selection of this option causes the test to end if this response was given.
Keys for end test—a set of keys having been assigned to the user during the current session that will cause the test to end.
End test text—additional text to be displayed after the test ends.
Force score—system defined score to be assigned to a user having selected this response, regardless of his previous score.
Delete—selection of the Delete option means that the response is to be deleted from the system for the current factor node, in the context of the current treatment.

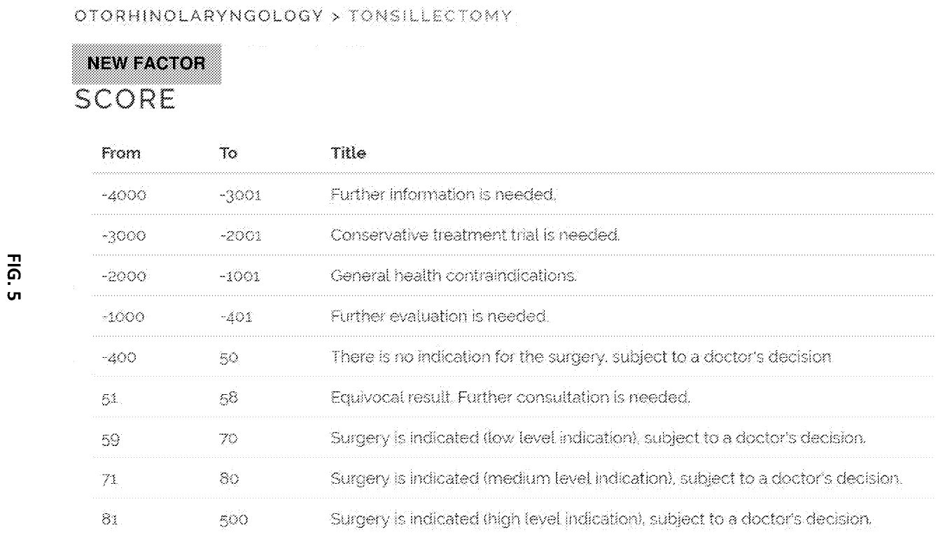

FIG. 5 shows an exemplary score table for an exemplary treatment-a tonsillectomy procedure.

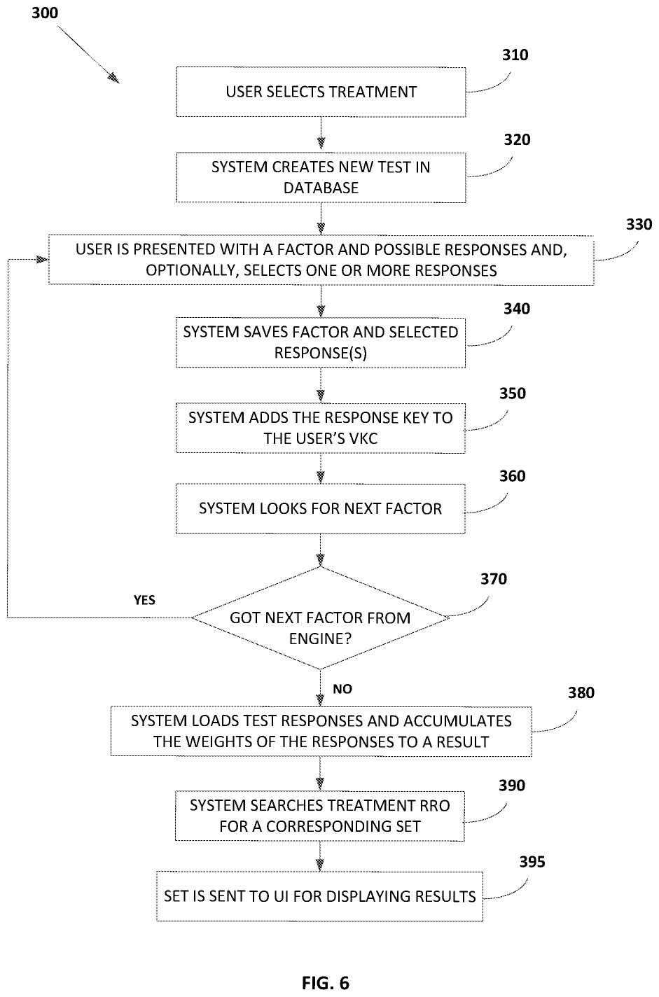

FIG. 6 is a flowchart 300 showing the steps taken during an exemplary session initiated by a user for obtaining an opinion regarding a medical treatment.

In step 310, the user selects a treatment from a given list of treatments and clicks “start test”.

In step 320, a new test is created in the system database 170, which may be a proprietary data repository. The test may include a timestamp, user info (IP etc.). A unique test ID is created.

During the test, the user typically sees the factors, ordered by their priorities. In step 330, the user is presented with a factor and, optionally, one or more possible responses to select from and selects the appropriate one or more responses.

The system then performs one or more of the following:
Saves the factor and selected response to a test database, with connection to the test ID (step 340).
Adds the response key (AK) (if exists) to the user's virtual key-chain (VKC) which comprises all current test response keys (step 350).

In step 360, the system checks in system's treatment's factors database for the next factor that matches some or all of the following criteria:
A factor with lower priority.
A factor that can be unlocked using the user's current VKC (looping through all existing keys and matching with factors' required keys).

In step 370, if the system found a new factor, it returns the new factor object to the user interface and loops back to step 330.

If no new factor was found, the system loads all the user's test responses from the database and accumulates the weight of the responses to a result (step 380).

It will be appreciated that the term ‘accumulates’ may include any mathematical operation or equation and the present invention is not limited to a simple sum operation.

In step 390, the system searches the treatment's RRO for the corresponding set, where result is between MINRV and MAXRV, and sends the found set to the user interface for results display (step 395).

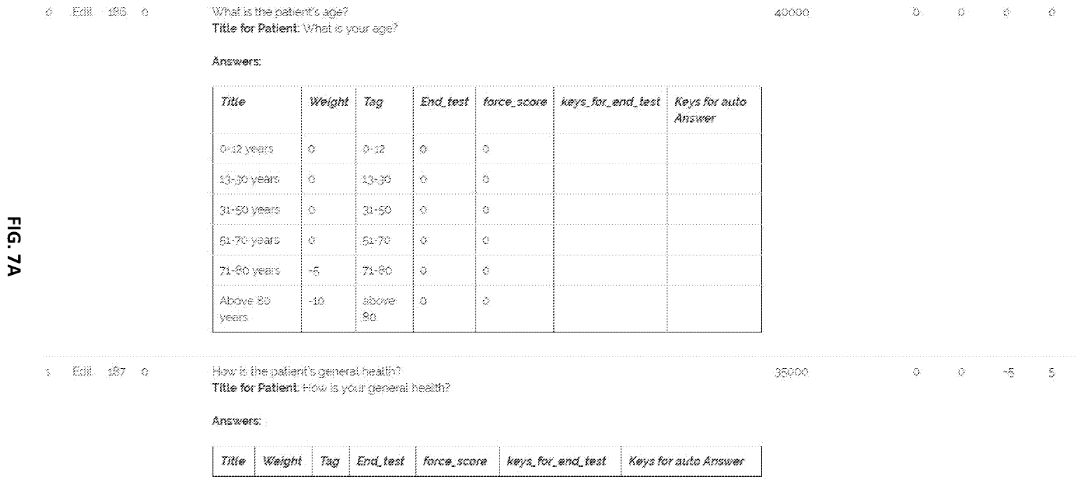

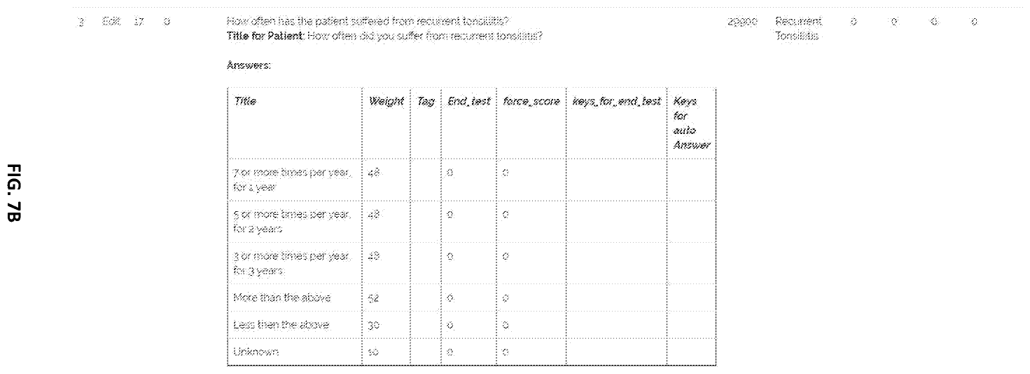

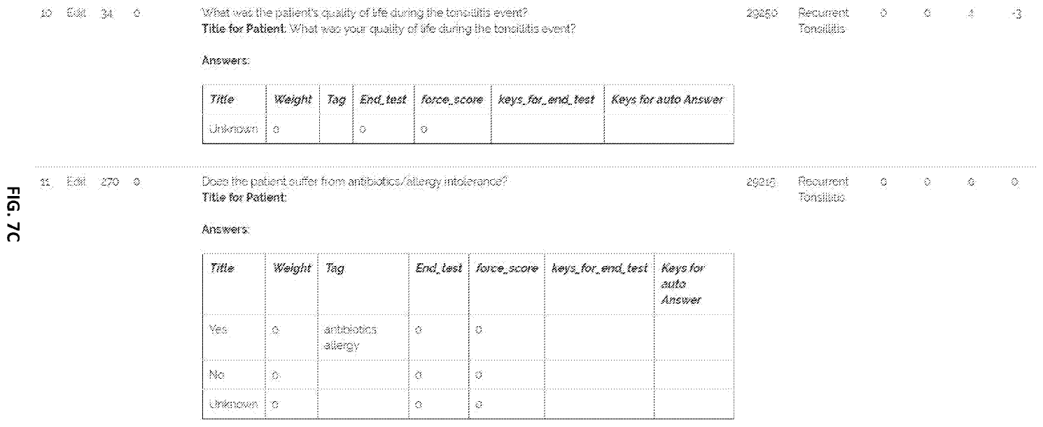

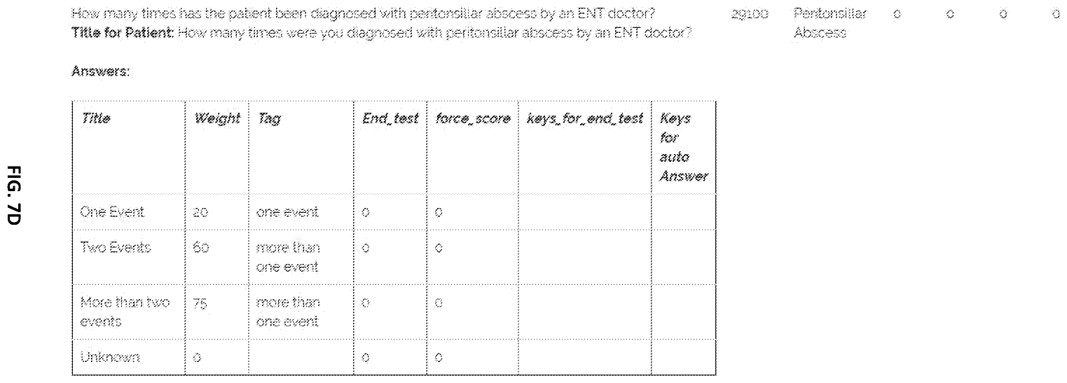

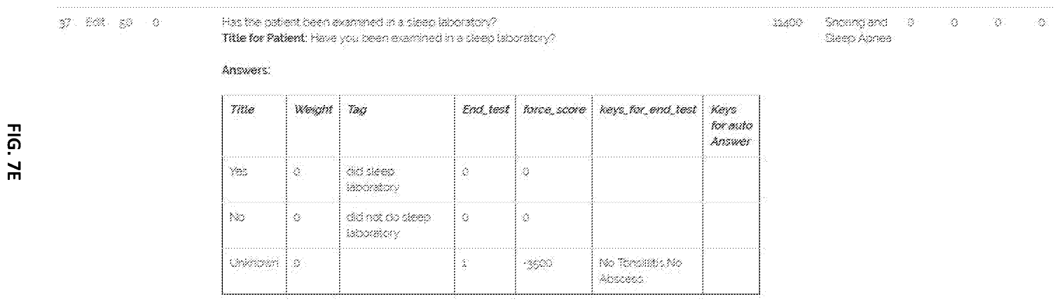

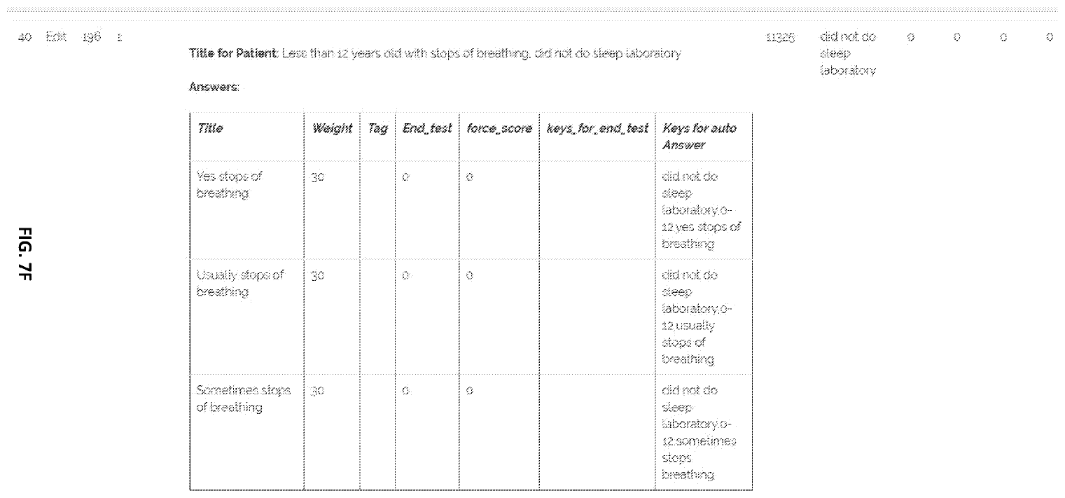

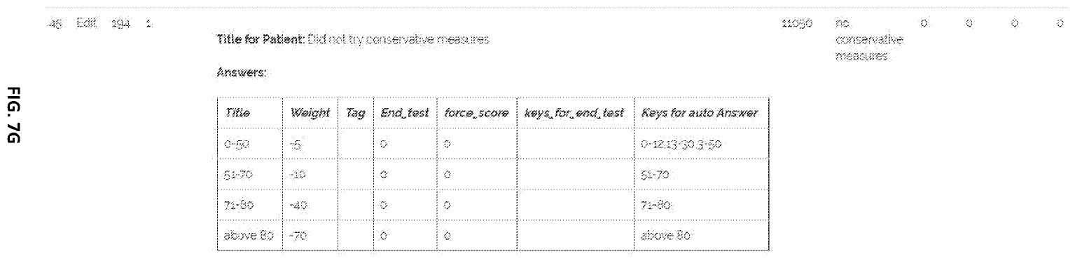

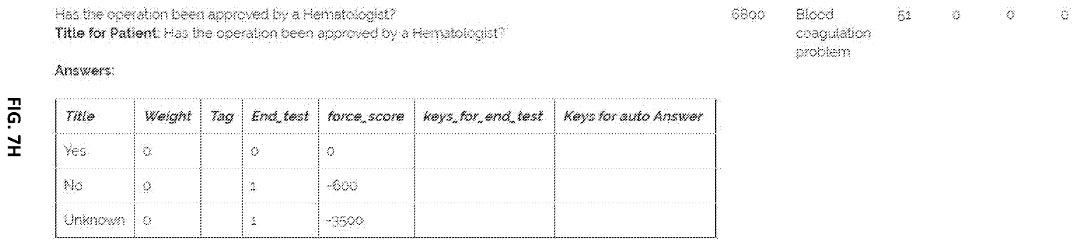

FIGS. 7A-7H provide an example of the following, for a single treatment:
A. factors for presentation to an end user e.g., via his cellphone or personal computer, all or any subset of which may be presented;
B. possible multiple choice responses thereto;
C. scoring of each possible response;

All typically developed by a human expert such as a medical doctor and/or using machine learning modules, based on one or more of: professionals and users' feedback, patients' databases and research and objective patient outcomes indicators.

The example is of a single treatment (Tonsillectomy) within a single specialty; in practice hundreds of treatments, or more, may be supported, within plural specialties.

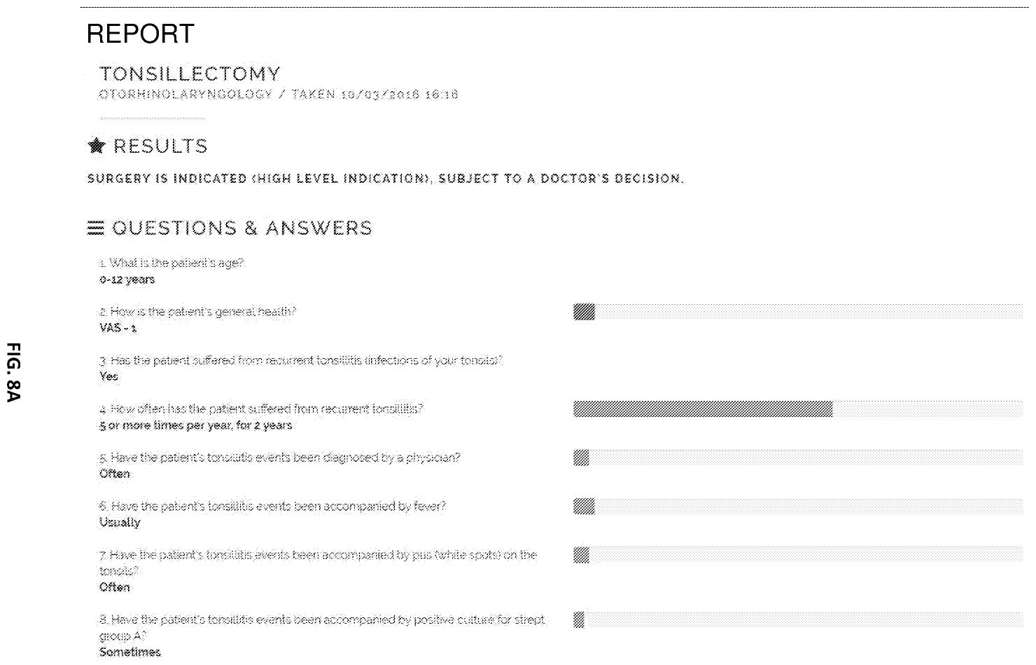

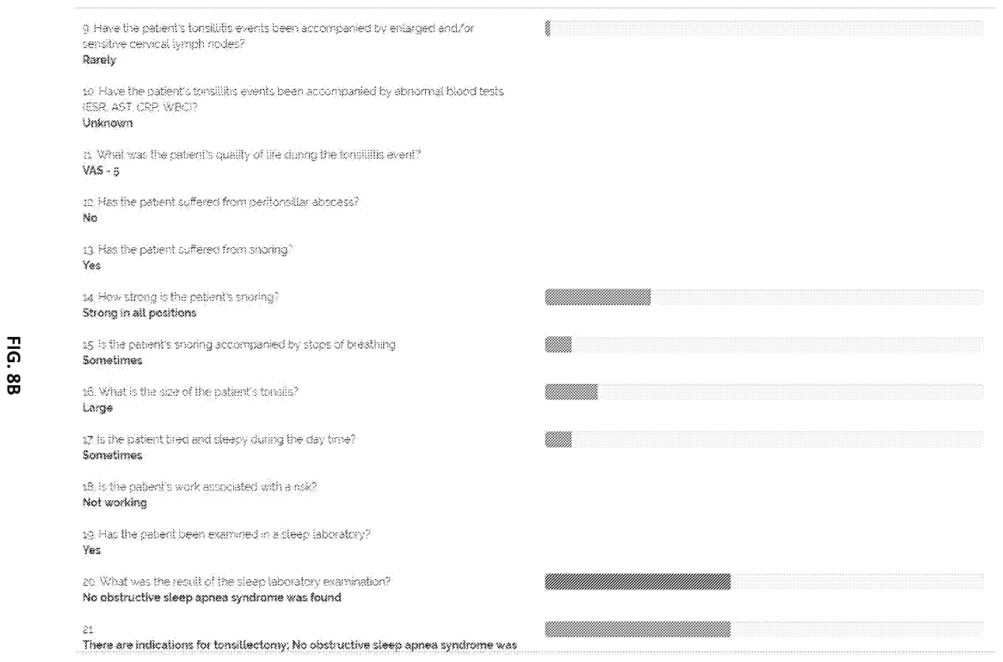

FIGS. 8A and 8B are exemplary reports provided by the system to the user at the end of a session.

The platform is typically configured to serve all or any subset of the following end-user types:
Patients considering a treatment.
Families and friends of patients considering a treatment.
Professionals using this as a tool in their daily practice.
Medical institutions.
Medical information providers.
Pharma companies
HMOs before approval of a certain treatment.
Insurance companies.
Policy decision makers in the medical field.
Medical legal entities.

Advantages may include:
a. Unnecessary and risky treatment shall be avoided.
b. Saving valuable resources for medical entities.
c. Improving the outcomes of medical treatments.
d. Patients are provided with knowledge for optimized decision-making concerning health risks and life saving dilemmas.
e. Professionals and patients are facilitated in asking the right factors and trying alternative treatments and doing required test before going under risky treatment.
f. The decision-making process before medical treatment is improved, transparent and documented.
g. Providing the right treatment to the right patient at the right time.
h. Dynamically and efficiently prioritizing waiting lists.

It is appreciated that the hierarchical flow and weights/points may be organized in any suitable logical relationship or structural combination. It is possible that different sets of factors and responses appear to each user and that different weights may be assigned for the same responses in different combinations, e.g., so as to yield a specific output for each individual end-user.

For some treatments, certain responses provided by an end-user may be deemed an absolute contraindication for the specific treatment and/or to the general/local anesthesia which are needed to conduct the treatment that was selected by the end-user, or may optionally result in the server presenting a recommendation for further information or examinations, another mode of treatment (e.g., conservative treatment) or another treatment entirely.

The tool can be presented in any digital platform and may provide end users with information on some or all of: treatments, descriptions, risks, statistics, tables, diagrams and drawings, along with automated decision making as described herein, thereby enhancing patients' ability to make more cautious medical decisions based on maximum information.

As mentioned above, the system of the present invention dynamically prioritizes patients' waiting lists, thereby enabling patients' receiving their treatment not only according the next available appointment but also as a function of various factors, such as, for example, their condition.

In order to provide such a dynamic prioritized waiting list, the machine learning module 130 collects, processes and analyzes at least some of: the treatment protocols, patients' outcomes and patients' profiles including patients' data and reports related to various treatments, received from at least one of the data mining and (NLP) module 120, the external databases 180, the big data resources 185 and the database 170 storing reports generated by the reports and statistics module 150 and finds connections and/or correlations between at least some of: the patients' profiles, treatment protocols, patients' outcomes as results of treatments and other factors such as time etc., thereby enabling to dynamically prioritize waiting lists

The patient's profile includes, but it not limited to include, patient's anonymous clinical history indicating treatment outcomes such as readmissions, complications, revisits, patients' and professionals' feedback and more.

The analysis, performed by the machine learning module 130, is constantly being updated and evolved, thereby enabling to provide more accurate results over time.

Hence, the method and system of the present invention enables to dynamically prioritize waiting lists.

With a solid expert system and a learned improvement in place, the system of the present invention uses real-world data to continuously improve the process using advanced learning machinery by allowing a greater breadth of measurements, “big data” scale of data processing using advanced machine learning techniques such as, for example, random forests or deep learning.

According to embodiments of the present invention, the system pursues three axes of data and ML progression in parallel:
1) Labeled Medical Records—Many health and insurance institutions have medical records that have already been labeled by the treating doctor. These records may be fed into the system, along with their labels. Machine learning techniques are then used to improve the labeling process.
2) Unlabelled Medical Records—Medical records from the practices of the present invention's experts or from medical institutions are also labeled by experts. Advance learning techniques are applied to much richer measurement spaces, taking into account a wealth of information that goes well beyond what can be captured by a hand-coded scenario.
3) User-Based Records—The system of the present invention is used to further improve the learning process. By using the system each user creates a real record, which can then be labeled by an expert. Further, user follow-up feedback can serve as a (noisy) automated labeling mechanism which can further improve the system.

Emphasis shall be on collecting objective indicators with regards to patient outcomes, such as, but not limited to, readmissions, complications, visits, patients' feedbacks and professionals' feedbacks.

Methods of finding insights between the data collected from historical data or the personalized chatbot, treatment protocols and the patient outcomes are implemented.

When a user has to schedule a treatment, the system of the present invention may assist him to schedule a personalized appointment.

It will be appreciated that the system of the present invention is not limited to using the reports generated by the present invention. Accordingly, any medical data related to the patient, may be used.

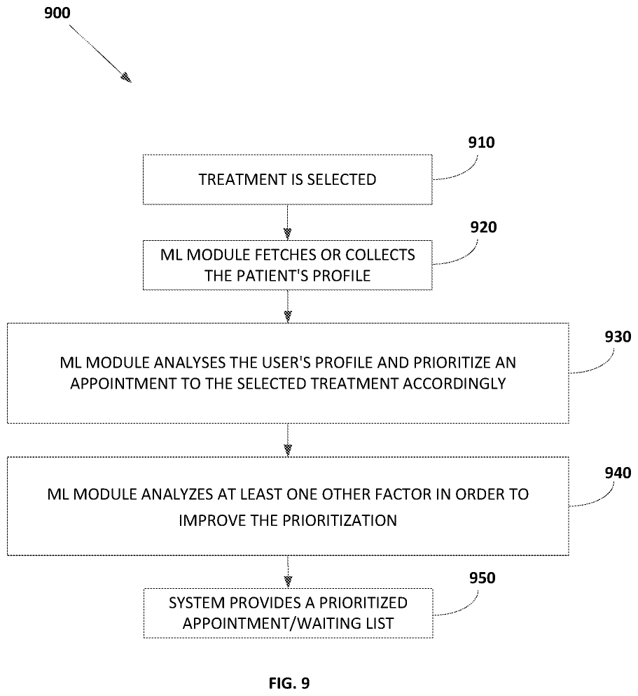

FIG. 9 is a flowchart 900 showing the process performed by the system of present invention when a personalized appointment has to be scheduled.

In step 910, the treatment is selected.

In step 920, the ML module fetches (if available) or collects the patient's profile.

The user's profile may include the user's data and reports related to various treatments the user has undergone in the past, received from at least one of the data mining and (NLP) module 120, the external databases 180, the big data resources 185 and the database 170 storing reports generated by the reports and statistics module 150.

In step 930, the ML module analyzes the user's profile and prioritizes an appointment to the selected treatment accordingly.

In step 940, the ML module may further analyze at least one of the following factors, in order to prioritize an appointment to the selected treatment:
1. Reversible indications—reversible factors which may influence the decision whether the patient is suitable for the selected treatment, e.g., weight.
2. None reversible factors—no indication for the selected treatment.
3. Timing.
4. Doctors' labeling—indications from doctors regarding the treatment urgency of each patient.
5. Using patients' outcomes to the selected treatment according to, for example, objective indicators and subjective indicators such as patients' and professionals' feedback.
6. Predetermined or selected factors, according to the patient.

In step 950, the system provides a prioritized appointment/waiting list.

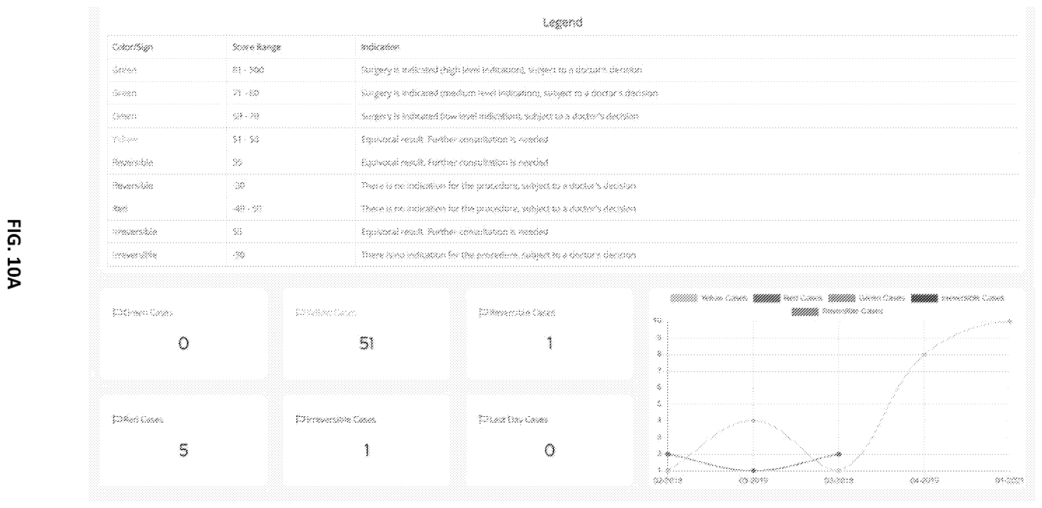

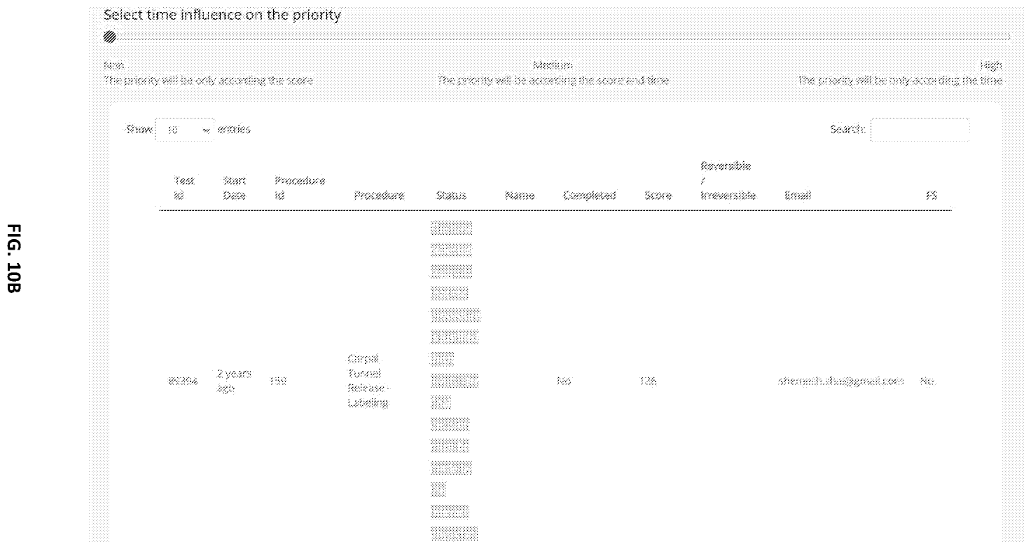

FIG. 10A shows an exemplary chart with the configured results to prioritize patients' waiting lists according to the ML predications and factors considered.

FIG. 10B shows exemplary detailed information for each case prioritized.

Features of the present invention, including method steps, which are described in the context of separate embodiments, may also be provided in combination in a single embodiment. Conversely, features of the invention, which are described for brevity in the context of a single embodiment or in a certain order may be provided separately, or in any suitable sub-combination or in a different order.

Any or all of computerized output devices or displays, processors, data storage and networks may be used as appropriate to implement any of the methods and apparatus shown and described herein.

It will be appreciated by persons skilled in the art that the present invention is not limited to what has been particularly shown and described hereinabove. Rather the scope of the present invention is defined by the appended claims and includes combinations and sub-combinations of the various features described hereinabove as well as variations and modifications thereof which would occur to persons skilled in the art upon reading the foregoing description.

1. An automated AI-based computerized system for dynamically prioritizing patients' waiting lists, comprising:
a system server configured to:
communicate with external medical sources;
store medical information from said external medical sources in a database; and
analyze said medical information using Natural Language Processing (NLP) and artificial intelligence tools;
said system server comprises a machine learning module configured to:
collect patients' profiles and at least one other factor;
analyze said patients' profiles and said at least one other factor; and
prioritize one of an appointment and a waiting list of a patient, according to said analysis of said patients' profiles and said at least one other factor.

2. The system of claim 1, wherein said system server comprises a reports and statistics module configured to generate personal reports to users following a chatbot session;
wherein said chatbot is configured to enable bi-directional communication with users seeking a prioritized appointment; and
wherein said communication comprises personally customized dynamic scenario comprising dynamic factors, responses and dynamic weights of said responses.

3. The system of claim 2, wherein said system server further comprises:
a data mining and NLP module;
a machine learning module;
an Application Program Interface (API) module configured to enable data retrieval from various external medical sources;
a management and control module;
at least one database;
a web application configured to provide users with an interactive platform for communicating with the system; and
a processing engine.

4. The system of claim 3, wherein said data mining and NLP module is configured to extract data from said external medical sources and transform it into an understandable structure for further use.

5. The system of claim 4, wherein said extracted data comprises data from patients' medical files; and wherein said extracted data is used for automatic labeling, for training said machine learning module.

6. (canceled)

7. The system of claim 3, wherein said machine learning module is configured to:
calibrate said dynamic weight of each response relevant to each treatment, by analyzing a large number of scenarios; and
calibrate the system using at least one of:
information mined from real medical files;
professionals' and/or patients' feedback after having undergone a treatment; and
scanning latest researches, statistics and publications by health organizations.

8. The system of claim 3, wherein said at least one database comprises:
patients' personal and medical information;
reports generated by said reports and statistics module;
a set of specific factors and possible responses for each treatment with complex relations, which are generated in advance by human experts and/or by machine learning modules; and
a set of dynamic weights associated with each response for different scenarios.

9. The system of claim 8, wherein at least one of said factors and possible responses and said dynamic weights are generated and updated by the system for each treatment, based on said data mining and NLP module and said machine learning module.

10. (canceled)

11. The system of claim 8, wherein said communicating with the system comprises presenting queries, receiving responses and receiving reports and recommendations.

12. The system of claim 8, wherein said processing engine is configured to:
select and present one factor at a time to said user;
receive a response to said factor;
assign a current dynamic weight to said user's response;
optionally assign a tag (key) to said user's response;
select next factor based on said user's response and one or more of said optional tags assigned to said user for previous responses; and
provide results to said reports and statistics module.

13. The system of claim 8, wherein said at least one database comprises, for each treatment:
a set of result range objects (RRO); and
a multi-dimensional tree of factor nodes (FN) and response objects (RO) for each factor node.

14. (canceled)

15. (canceled)

16. (canceled)

17. The system of claim 1, wherein said at least one other factor is selected from the group consisting of: reversible indications, non-reversible factors, timing, doctors' labeling, patients' outcomes including objective and subjective indicators to said selected treatment and predetermined or selected factors, according to said patient.

18. An automated method of dynamically prioritizing patients' waiting lists, comprising:
retrieving medical data from medical sources and storing said retrieved data;
analyzing said medical data using Natural Language Processing (NLP) and artificial intelligence tools;
collecting patients' profiles and at least one other factor;
analyzing said patients' profiles and said at least one other factor; and
prioritizing one of an appointment and a waiting list of a patient, according to said analysis of said patients' profiles and said at least one other factor.

19. The method of claim 18, wherein said collecting patients' profiles comprises:
computerizing a set of dynamic factors and possible responses in a hierarchic data structure with complex relations and a different dynamic weight for each response in the context of each treatment and scenario, said dynamic weights calculated by analyzing, using an artificial intelligence module, said treatment data;
receiving from a user, a request to prioritize an appointment;
providing a personalized customized dynamic scenario to said user, said scenario dynamically created, using said artificial intelligence module, according to said treatment, responses of said user, and said dynamic weights assigned to said user's responses to previous factors in said scenario;
computing a relative indication including providing a positive impact if a specific response and its dynamic weight, supports said treatment, and a negative impact if a specific response and its dynamic weight, negates said treatment according to said response's relative importance and impact on a decision to conduct said treatment; and
generating a specific personalized report for said user based on said treatment and including a relative indication for said treatment.

20. The method of claim 19, wherein said artificial intelligence module comprises a logic base derived from experience of human experts, statistical information and analysis of published studies and machine learning modules.

21. The method of claim 19, further comprising assigning at least one key (tag) to said user's response.

22. The method of claim 21, wherein dynamically creating said scenario comprises one of: selecting a next factor according to keys accumulated so far in said scenario; and ending said scenario according to keys accumulated so far in said scenario.

23. (canceled)

24. The method of claim 19, wherein said user is a patient and wherein said specific personalized report comprises at least one of: data related to said treatment, statistics, risks and questions to ask their physicians before going under said treatment.

25. The method of claim 19, wherein said user is a medical professional and wherein said specific personalized report comprises at least one of: data on at least some of treatments, statistics, risks and other factors with regards to treatments decision making process in the daily practice.

26. (canceled)

27. An automated method of dynamically prioritizing patients' waiting lists, comprising:
selecting, by a patient, an appointment to be prioritized;
fetching or collecting a patient's profile of said patient and other patients' profiles;
analyzing said patient's profile, said other patients' profiles and at least one other factor;
prioritizing one of an appointment and a waiting list of said patient, according to said patient's profile, said other patients' profiles and said at least one other factor.

28. (canceled)
